(19) United States
(12) Patent Application Publication
(10) Pub. No.: US 2002/0082594 A1
(43) Pub. Date: Jun. 27, 2002

(54) Injectable biomaterial and methods for use thereof

(76) Inventors: Cary Hata, Tustin, CA (US); Thomas Witzel, Laguna Niguel, CA (US)

Correspondence Address: Cary Hata, 2500 San Simon, Tustin, CA, 92782, US

(21) Appl. No.: 10/083,264

(22) Filed: Oct. 22, 2001

Related U.S. Application Data
(63) Continuation-in-part of application No. 09/410,902, filed on Oct. 2, 1999, now Pat. No. 6,306,133.

(51) Int. Cl. A61B 18/18

(52) U.S. Cl. 606/41

(57) ABSTRACT

A delivery system and methods for repairing an annular organ structure comprising injecting a heat shapeable biomaterial formulated for in vivo administration by injection via a delivery system at a site of the valvular annulus defect; and applying heat sufficient to shape and immobilize the biomaterial at about the annulus defect, and optionally to shape tissue surrounding the annulus defect.

### CROSS REFERENCE TO RELATED APPLICATION

This application is a continuation-in-part of application Ser. No. 09/410,902 filed Oct. 2, 1999, now U.S. Pat. No. 6,306,133, which is incorporated by reference herein in its entirety.

### FIELD OF THE INVENTION

This invention generally relates to systems and methods for applying energy to a patient for medical purposes such as shrinking and immobilizing an injectable biomaterial. More particularly, the invention relates to a catheter system that penetrates the tissue of a valvular annulus in order to inject and stabilize shapeable biomaterial adapted for repairing an annular organ structure defect of the patient.

### BACKGROUND OF THE INVENTION

The circulatory system consists of a heart and blood vessels. In its path through the heart, the blood encounters four valves. The valve on the right side that separates the right atrium from the right ventricle has three cusps and is called the tricuspid valve. It closes when the ventricle contracts during a phase known as systole and it opens when the ventricle relaxes, a phase known as diastole.

The pulmonary valve separates the right ventricle from the pulmonary artery. It opens during systole, to allow the blood to be pumped toward the lungs, and it closes during diastole to keep the blood from leaking back into the heart from the pulmonary artery. The pulmonary valve has three cusps, each one resembling a crescent and it is also known as a semi-lunar valve.

The mitral valve, so named because of its resemblance to a bishop's mitre, is in the left ventricle and it separates the left atrium from the ventricle. It opens during diastole to allow the blood stored in the atrium to pour into the ventricle, and it closes during systole to prevent blood from leaking back into the atrium. The mitral valve and the tricuspid valve differ significantly in anatomy. The annulus of the mitral valve is somewhat D-shaped whereas the annulus of the tricuspid valve is more nearly circular.

The fourth valve is the aortic valve. It separates the left ventricle from the aorta. It has three semi-lunar cusps and it closely resembles the pulmonary valve. The aortic valve opens during systole allowing a stream of blood to enter the aorta and it closes during diastole to prevent any of the blood from leaking back into the left ventricle.

In a venous circulatory system, a venous valve is to prevent the venous blood from leaking back into the upstream side so that the venous blood can return to the heart and the lungs for blood oxygenating purposes.

Clinical experience has shown that repair of a valve, either a heart valve or a venous valve, produces better long-term results than does valve replacement. Valve replacement using a tissue valve suffers long-term calcification problems. On the other hand, anticoagulation medicine, such as heparin, is required for the life of a patient when a mechanical valve is used in valve replacement. The current technology for valve repair or valve replacement requires an expensive open-heart surgery that needs a prolonged period of recovery. A less invasive catheter-based valve repair technology becomes an unmet clinical challenge.

The effects of valvular dysfunction vary. Mitral regurgitation has more severe physiological consequences to the patient than does tricuspid valve regurgitation. In patients with valvular insufficiency it is an increasingly common surgical practice to retail the natural valve, and to attempt to correct the defects. Many of the defects are associated with dilation of the valve annulus. This dilatation not only prevents competence of the valve but also results in distortion of the normal shape of the valve orifice or valve leaflets. Remodeling of the annulus is therefore central to most reconstructive procedures for the mitral valve.

As a part of the valve repair it is either necessary to diminish or constrict the involved segment of the annulus so that the leaflets may coapt correctly on closing, or to stabilize the annulus to prevent post-operative dilatation from occurring. The current open-heart approach is by implantation of a prosthetic ring, such as a Cosgrove Ring or a Carpentier Ring, in the supra annular position. The purpose of the ring is to restrict and/or support the annulus to correct and/or prevent valvular insufficiency. In tricuspid valve repair, constriction of the annulus usually takes place in the posterior leaflet segment and in a small portion of the adjacent anterior leaflet.

Various prostheses have been described for use in conjunction with mitral or tricuspid valve repair. The ring developed by Dr. Alain Carpentier (U.S. Pat. No. 3,656,185) is rigid and flat. An open ring valve prosthesis as described in U.S. Pat. No. 4,164,046 comprises a uniquely shaped open ring valve prosthesis having a special velour exterior for effecting mitral and tricuspid annuloplasty. The fully flexible annuloplasty ring could only be shortened in the posterior segment by the placement of placating sutures. John Wright et al. in U.S. Pat. No. 5,674,279 discloses a suturing ring suitable for use on heart valve prosthetic devices for securing such devices in the heart or other annular tissue. All of the above valve repair or replacement requires an open-heart operation which is costly and exposes a patient to higher risk and longer recovery than a catheter-based less invasive procedure.

Moderate heat is known to tighten and shrink the collagen tissue as illustrated in U.S. Pat. No. 5,456,662 and U.S. Pat. No. 5,546,954. It is also clinically verified that thermal energy is capable of denaturing the tissue and modulating the collagenous molecules in such a way that treated tissue becomes more resilient (“The Next Wave in Minimally Invasive Surgery” MD&DI pp. 36-44, August 1998). Therefore, it becomes imperative to treat the inner walls of an annular organ structure of a heart valve, a valve leaflet, chordae tendinae, papillary muscles, and the like by shrinking/tightening techniques. The same shrinking/tightening techniques are also applicable to stabilize injected biomaterial to repair the defect annular organ structure, wherein the injectable biomaterial is suitable for penetration and heat treatment.

One method of reducing the size of tissues in situ has been used in the treatment of many diseases, or as an adjunct to surgical removal procedures. This method applies appropriate heat to the tissues, and causes them to shrink and tighten. It can be performed on a minimal invasive fashion, which is often less traumatic than surgical procedures and may be the only alternative method, wherein other procedures are unsafe or ineffective. Ablative treatment devices have an advantage because of the use of a therapeutic energy that is rapidly dissipated and reduced to a non-destructive level by conduction and convection, to other natural processes.

Radiofrequency (RF) therapeutic protocol has been proven to be highly effective when used by electrophysiologists for the treatment of tachycardia; by neurosurgeons for the treatment of Parkinson's disease; by otolaryngologist for clearing airway obstruction and by neurosurgeons and anesthetists for other RF procedures such as Gasserian ganglionectomy for trigeminal neuralgia and percutaneous cervical cordotomy for intractable pains. Radiofrequency treatment, which exposes a patient to minimal side effects and risks, is generally performed after first locating the tissue sites for treatment. Radiofrequency energy, when coupled with a temperature control mechanism, can be supplied precisely to the device-to-tissue contact site to obtain the desired temperature for treating a tissue or for effecting the desired shrinking of the injected biomaterial adapted to immobilize the biomaterial in place. It is another object to shape the injected biomaterial along with the tissue surrounding the injected biomaterial.

Therefore, there is a clinical need to have a less invasive catheter-based approach for repairing an annular organ structure of a heart valve, a valve leaflet, chordae tendinae, papillary muscles, and the tissue defect by using high frequency energy for reducing and/or shrinking an injected biomaterial along with the host tissue mass for tightening and stabilizing the dilated tissue adjacent a valvular annulus.

### SUMMARY OF THE INVENTION

In general, it is an object of the present invention to provide a delivery system and methods for repairing an annular organ structure of a heart valve, an annular organ structure of a venous valve, a valve leaflet, chordae tendinae, papillary muscles, and the like.

It is another object of the present invention to provide a delivery system and methods by using high frequency current for tissue treatment or repairing.

It is still another object to provide a delivery catheter system that penetrates the tissue of a valvular annulus in order to tighten and stabilize an annular organ structure.

It is a preferred object to provide a method for repairing a valvular annulus defect comprising injecting a heat shapeable biomaterial formulated for in vivo administration by injection via a delivery system at a site of the valvular annulus defect; and applying heat sufficient to shape the biomaterial and immobilize the biomaterial at about the annulus defect.

It is another object of the invention to provide a method for repairing a tissue defect comprising: injecting a heat shapeable biomaterial formulated for in vivo administration by injection via a percutaneous delivery system at a site of the tissue defect; and applying heat to the biomaterial and a portion of the tissue defect adapted for shaping the biomaterial, the heat being below a temperature sufficient for effecting crosslinking of the biomaterial and the portion of the tissue defect.

It is still another object of the present invention to provide a delivery system and methods for providing high frequency current energy to the tissue/organ at or adjacent a heart valve structure.

In one embodiment, the method comprises: percutaneously introducing the delivery system through a blood vessel to a site of the valvular annulus or introducing the delivery system through a thoroscopy port into a heart or injecting the heat shapeable biomaterial during an open heart surgery; positioning the tissue-contactor ring of the catheter shaft on the inner wall of the valvular annulus; advancing the needle electrode element for penetrating the needle electrode element into a tissue of the valvular annulus; injecting heat shapeable biomaterial at the site of the valvular annulus defect; and applying high frequency current through the electrical conductor means to the needle electrode element for repairing the valvular annulus defect.

### BRIEF DESCRIPTION OF THE DRAWING

Additional objects and features of the present invention will become more apparent and the invention itself will be best understood from the following Detailed Description of the Exemplary Embodiments, when read with reference to the accompanying drawings.

FIG. 1 is an overall view of a delivery system having a flexible tissue-contactor means and a needle electrode means at its distal tip section constructed in accordance with the principles of the present invention, wherein the needle electrode means is also configured for delivering injectable biomaterial into the tissue defect.

FIG. 2 is a close-up view of the distal tip section of the delivery system comprising a retracted tissue-contactor means and a retracted needle electrode means at a non-deployed state.

FIG. 3 is a close-up view of the distal tip section of the delivery system comprising a deployed tissue-contactor means and a retracted needle electrode means.

FIG. 4 is a front cross-sectional view; section A-A of FIG. 3, of the distal tip section of a delivery system comprising a deployed tissue-contactor means.

FIG. 5 is a close-up view of the distal tip section of the delivery system comprising a deployed tissue-contactor means and a deployed needle electrode means at a fully deployed state, wherein the needle electrode means is connected to a shapeable biomaterial source for injecting the biomaterial into the tissue defect.

FIG. 6 is a front cross-sectional view, section B-B of FIG. 5, of the distal tip section of a delivery system comprising a deployed tissue-contactor means and a deployed needle electrode means.

FIG. 7 is a simulated view of one embodiment applying a catheter-based delivery system of the present invention in contact with the tissue of an annular organ structure.

### DETAILED DESCRIPTION OF THE EXEMPLARY EMBODIMENTS

The following descriptions of the preferred embodiment of the invention are exemplary, rather than limiting, and many variations and modifications are within the scope of the invention.

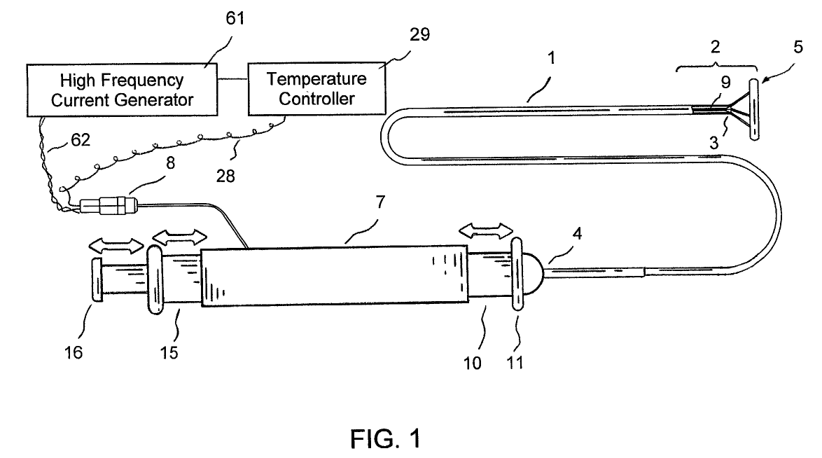

It is one object of the present invention to provide a method for repairing a valvular annulus defect comprising injecting a heat shapeable biomaterial formulated for in vivo administration by injection via a delivery system at a site of the valvular annulus defect; and applying heat sufficient to shape the biomaterial and immobilize the biomaterial at about the annulus defect. FIG. 1 shows an overall view of a catheter-based delivery system having a flexible tissue-contactor means and a needle electrode means at its distal tip section constructed in accordance with the principles of the present invention. A delivery system constructed in accordance with the principles of the present invention comprises a flexible catheter shaft 1 having a distal tip section 2, a distal end 3, a proximal end 4, and at least one lumen 14 extending therebetween.

In one embodiment, the catheter system comprises a flexible, relatively semi-rigid tissue-contactor means 5 located at the distal tip section 2 and inside the at least one lumen 14 of the catheter shaft 1 for contacting an inner wall 51 of an annular organ structure 52 when deployed. The tissue-contactor means 5 is deployable out of the at least one lumen 14 by a tissue-contactor deployment mechanism 15 located at a handle 7. The tissue-contactor means 5 is preformed to have an appropriate shape compatible with the inner wall 51 of the annular organ structure 52. The tissue-contactor means 5 may be selected from the group consisting of a circular ring, a D-shaped ring, a kidney-shaped ring, an oval ring, and other round-shaped mass.

A handle 7 is attached to the proximal end 4 of the catheter shaft 1. The handle comprises the tissue-contactor deployment mechanism 15 and an electrode deployment means 16 for advancing a needle electrode means 9 out of the tissue-contactor means 5.

A connector 8 secured at the proximal end of the catheter system, is part of the handle section 7. The handle has one optional steering mechanism 10. The steering mechanism 10 is to deflect the distal tip section 2 of the catheter shaft 1 for catheter maneuvering and positioning. By pushing forward the front plunger 11 of the handle 7, the distal tip section 2 of the catheter shaft deflects to one direction. By pulling back the front plunger 11, the tip section returns to its neutral position. In another embodiment, the steering mechanism 10 at the handle 7 comprises means for providing a plurality of deflectable curves on the distal tip section 2 of the catheter shaft 1.

The catheter system also comprises a high frequency current generator 61, wherein an electrical conductor means 62 for transmitting high frequency current to the needle electrode means 9 is provided. The high frequency current may be selected from a group consisting of radiofrequency current, microwave current and ultrasound current.

The method may comprise percutaneously introducing the delivery system through a blood vessel to a site of the valvular annulus or introducing the delivery system through a thoroscopy port into a heart or injecting the heat shapeable biomaterial during an open heart surgery.

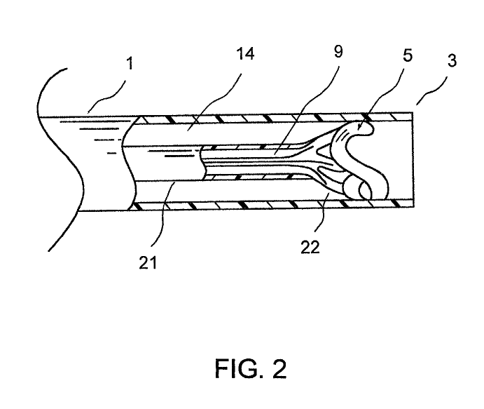

FIG. 2 shows a close-up view of the distal tip section 2 of the catheter system comprising a retracted tissue-contactor means 5 and a retracted needle electrode means 9 at a non-deployed state. Both the tissue-contactor means and the needle electrode means are retractable to stay within the at least one lumen 14. This non-deployed state is used for a catheter to enter into and to withdraw from the body of a patient. The tissue-contactor means is preformed and flexible enough so that it can easily retracted into the catheter lumen 14.

The tissue-contactor means 5 may be made of a biocompatible material selected from the group consisting of silicone, latex, polyurethane, fabric, and a combination thereof. Reinforced substrate, such as mesh, wire, fiber, and the like, may be added to the tissue-contactor means 5 to make the tissue-contactor means semi-rigid so that when it is deployed, adequate pressure is exerted to the surrounding tissue for stabilizing its placement.

The catheter system comprises a needle electrode means 9 located at or within the flexible tissue-contactor means 5 for penetrating into a tissue, such as an inner wall 51, wherein the needle electrode means 9 is deployable out of the tissue-contactor means 5 in a manner essentially perpendicular to a longitudinal axis of the catheter shaft 1 when the needle electrode means is deployed. In another preferred embodiment, the angle of the needle electrode against a tissue may be any suitable angle from 30 degrees to 150 degrees in reference to a longitudinal axis of the catheter shaft for effective tissue penetration.

The needle electrode means 9 may comprise a plurality of needle electrodes 9A, 9B, 9C that are preshaped to be essentially perpendicular to a longitudinal axis of the catheter shaft 1 when deployed. The high frequency current may be delivered to each of the plurality of needle electrodes 9A, 9B, 9C in a current delivery mode selected from the group consisting of individual delivery mode, pulsed delivery mode, sequential delivery mode, and simultaneous delivery mode. The needle electrode means 9 may be made of a material selected from the group consisting of platinum, iridium, gold, silver, stainless steel, tungsten, Nitinol, and other conducting material. The needle electrode means 9 is connected to an electrode deployment means 16 at the handle 7 for advancing one or more needles of the needle electrode means 9 out of the tissue-contactor means 5. This electrode deployment means may include various deployment modes of a single needle electrode deployment, a plurality of needle electrodes deployment or all needle electrodes simultaneous deployment.

The tissue-contactor means 5 in this invention is defined as a flexible semi-rigid element adapted for contacting an inner wall of an annular organ structure of a patient and is also preformed to have an appropriate shape compatible with the inner wall of the annular organ structure. The tissue-contactor means may comprise a plurality of grooves or internal channels 25 so that a needle electrode of the needle electrode means is able to deploy out of and retract into the tissue contactor means with minimal frictional resistance.

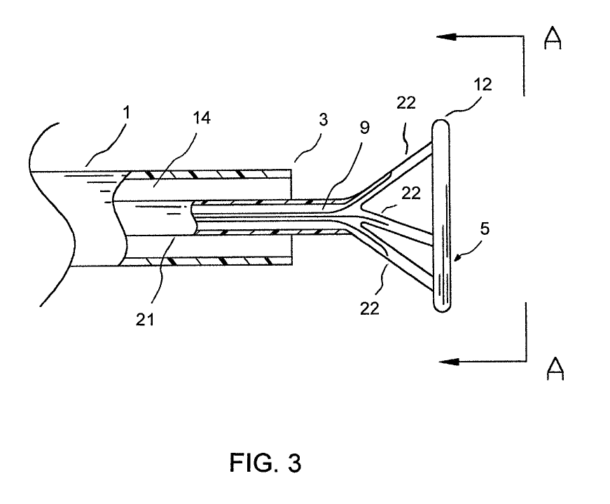

FIG. 3 shows a close-up view of the distal tip section 2 of the present catheter system comprising a deployed tissue-contactor means 5 and a retracted needle electrode means 9. The outer diameter of the deployed tissue-contactor means 5 is optionally larger than the outer diameter of the catheter shaft 1 so that the outer rim 12 of the deployed tissue-contactor means may stably stay on the inner wall of the annular organ structure. A supporting member 21 along with a plurality of auxiliary supporting members 22 on the distal end of the supporting member 21 form a connecting means for connecting the tissue-contactor means 5 to the tissue-contactor deployment mechanism 15 that is located on the handle 7. The supporting member 21 and the auxiliary supporting members 22 are located within the at least one lumen 14 and have torque transmittable property and adequate rigidity to deploy the tissue-contactor means 5.

The needle electrode is preferably made of conductive material, while the surfaces of the catheter shaft 1, conducting wires 62, the supporting member 21, and the auxiliary supporting members 22, are preferably covered with an insulating material or insulated.

In one preferred embodiment, the needle electrode is hollow with a fluid conduit connected to an external fluid source having a fluid injection mechanism. By “fluid” is meant an injectable shapeable biomaterial that is formulated for in vivo administration by injection via a delivery system at a site of the valvular annulus defect or tissue defect. By “tissue defect” is meant vulnerable plaque, calcified tissue, valvular annulus defect, or other lesions of atherosclerosis.

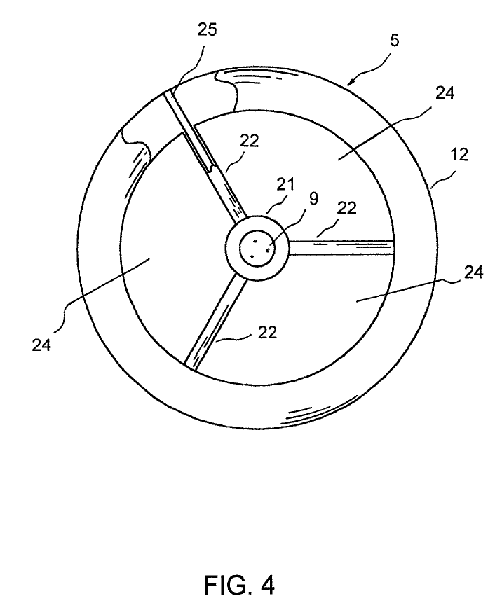

FIG. 4 shows a front cross-sectional view; section A-A of FIG. 3, of the distal tip section of a catheter system comprising a deployed tissue-contactor means 5. The tissue-contactor means 5 may comprise a plurality of open channels 24, pores and the like for a fluid or blood to pass from a proximal end of the tissue-contactor means 5 to a distal end of the tissue-contactor means.

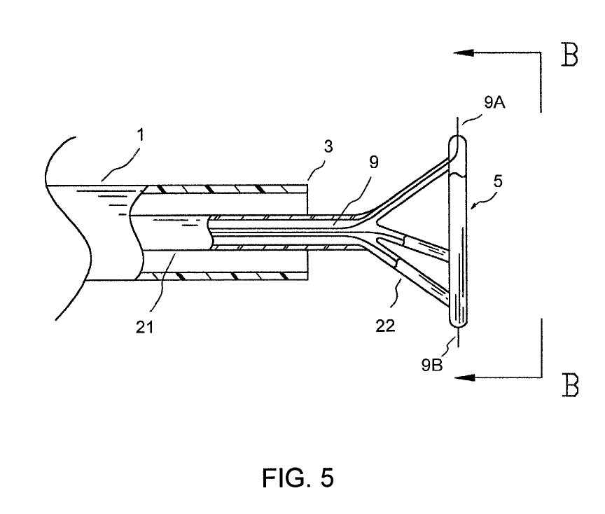

FIG. 5 shows a close-up view of the distal tip section 2 of the present catheter system comprising a deployed tissue-contactor means 5 and a deployed needle electrode means 9 at a fully deployed state. The fully deployed state is used for delivery of high frequency current energy to the needle electrode means 9 and subsequently to the contact tissue for repairing the annular organ structure. The delivery of high frequency current to each of the needle electrodes may go through a splitter or other mechanism. The needle electrode means is preformed so that when deployed, the needle electrodes are in a manner essentially perpendicular to a longitudinal axis of the catheter shaft 1 for effective thermal therapy.

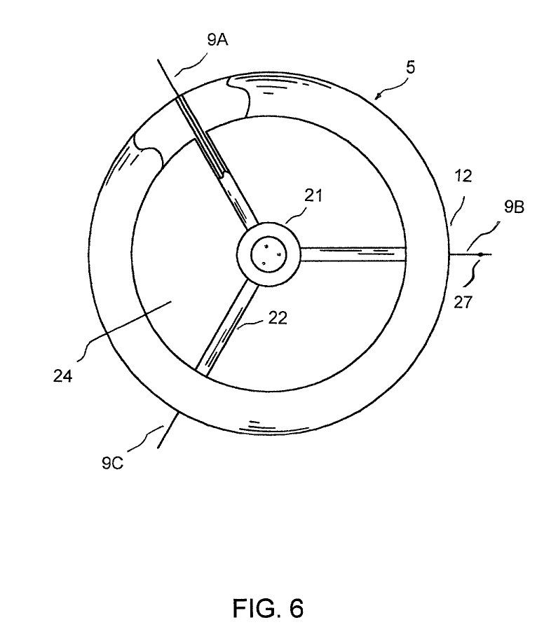

FIG. 6 shows a front cross-sectional view, section B-B of FIG. 5, of the distal tip section 2 of a catheter system comprising a deployed tissue-contactor means 5 and a deployed needle electrode means 9. The tips of the needle electrodes 9A, 9B, and 9C extend out of the rim 12 of the tissue-contactor means 5 and penetrate into a tissue for energy delivery.

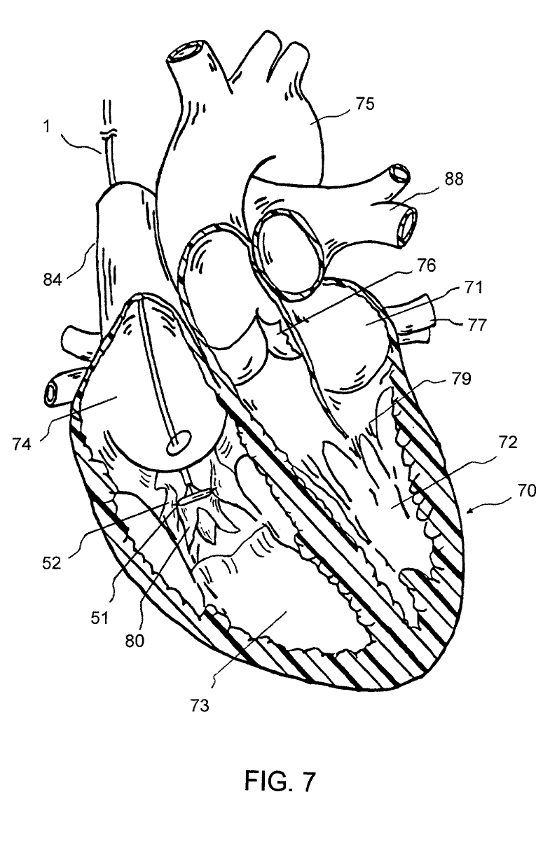

FIG. 7 shows a simulated view of the catheter system of the present invention in contact with the tissue of an annular organ structure 52. The heart 70 has a left atrium 71, a left ventricle 72, a right ventricle 73, and a right atrium 74. Aorta 75 connects with the left ventricle 72 and contains an aorta valve 76. Pulmonary artery 77 connects with the right ventricle 73 through a pulmonary valve. Left atrium 71 communicates with the left ventricle 72 through a mitral valve 79. The right atrium 74 communicates with the right ventricle 73 through a tricuspid valve 80. Oxygenated blood is returned to the heat 70 via pulmonary veins 88. In a perspective illustration, a catheter is inserted into the right atrium 74 and is positioned on the inner wall 51 of the tricuspid valve 80. The leaflets of the tricuspid valve 80 open toward the ventricle side. Blood returned from the superior vena cava 84 and the inferior vena cava flows into the right atrium 74. Subsequently, blood flows from the right atrium 74 to the right ventricle 73 through the tricuspid valve 80. Therefore, the tissue-contactor means 5 of the catheter shaft 1 does not interfere with the leaflet movement during the proposed less invasive thermal therapy of the invention.

In a preferred embodiment, a method for operating a delivery system of the present invention for repairing a valvular annulus, the method comprises (a) percutaneously introducing the delivery system through a blood vessel to a site of the valvular annulus or introducing the delivery system through a thoroscopy port into a heart or injecting the heat shapeable biomaterial during an open heart surgery; (b) positioning the tissue-contactor ring of the catheter shaft on the inner wall of the valvular annulus; (c) advancing the needle electrode element for penetrating the needle electrode element into a tissue of the valvular annulus; (d) injecting heat shapeable biomaterial at the site of the valvular annulus defect; and (e) applying high frequency current through the electrical conductor means to the needle electrode element for repairing the valvular annulus defect.

In another preferred embodiment, a method for operating a catheter system for repairing a tissue of a heart valve, the catheter system comprises a flexible catheter shaft having a distal tip section, a distal end, a proximal end, and at least one lumen extending between the distal end and the proximal end; an electrode means located at the distal tip section of the catheter shaft for contacting the tissue of the heart valve; a handle attached to the proximal end of the catheter shaft, wherein the handle has a cavity; and a high frequency current generator, wherein an electrical conductor means for transmitting high frequency current to the electrode means is provided. The method comprises (a) percutaneously introducing the catheter system through a blood vessel to the tissue of the heart valve; (b) positioning the electrode means of the catheter system at the tissue of the heart valve; and (c) applying high frequency current through the electrical conductor means to the electrode means for repairing the heart valve.

The tissue of the heart valve in the procedures may be selected from the group consisting of valvular annulus, chordae tendinae, valve leaflet, and papillary muscles. The high frequency current in the procedures may be selected from the group consisting of radiofrequency current, microwave current, and ultrasound current.

A temperature sensor 27, either a thermocouple type or a thermister type, is constructed at the proximity of the needle electrode 9B (shown in FIG. 6) to measure the tissue contact temperature when high frequency energy is delivered. A temperature sensing wire 28 from the thermocouple or thermister is connected to one of the contact pins of the connector 8 and externally connected to a transducer and to a temperature controller 29. The temperature reading is thereafter relayed to a closed-loop control mechanism to adjust the high frequency energy output. The high frequency energy delivered is thus controlled by the temperature sensor reading or by a pre-programmed control algorithm.

This invention discloses a method for repairing a valvular annulus defect, the method comprising injecting a heat shapeable biomaterial formulated for in vivo administration by injection via a catheter system at a site of the valvular annulus defect; and applying heat sufficient to shape the biomaterial and immobilize the biomaterial at about the annulus defect.

The term “shapeable biomaterial” as used herein is intended to mean any biocompatible material that changes its shape, size, or configuration at an elevated temperature without significantly affecting its composition or structure. The shaping of a shapeable biomaterial is usually accomplished by applying moderate energy. For example, a crosslinked material is structurally different from a non-crosslinked counterpart and is not considered as a shaped material. The elevated temperature in this invention may range from about 39° C. to about 45° or higher, wherein the heat is below a temperature for effecting crosslinking of the biomaterial.

The biomaterial may comprise a matrix of collagen, a connective tissue protein comprising naturally secreted extracellular matrix, a heat shapeable polymer, or the like.

The term “matrix of collagen” as used herein is intended to mean any collagen that is injectable through a suitable applicator, such as a catheter, a cannula, a needle, a syringe, or a tubular apparatus. The matrix of collagen as a shapeable biomaterial of the present invention may comprise collagen in a form of liquid, colloid, semi-solid, suspended particulate, gel, paste, combination thereof, and the like. Devore in PCT WO 00/47130 discloses injectable collagen-based system defining matrix of collagen, entire disclosure of which is incorporated herein by reference.

The shapeable biomaterial may further comprise a pharmaceutically acceptable carrier for treating the annulus defect and a drug is loaded with the pharmaceutically acceptable carrier, wherein the drug is selected from a group consisting of an anti-clotting agent, an anti-inflammatory agent, an anti-virus agent, an antibiotics, a tissue growth factor, an anesthetic agent, a regulator of angiogenesis, a steroid, and combination thereof.

The connective tissue protein comprising naturally secreted extracellular matrix as a shapeable biomaterial of the present invention may be biodegradable and has the ability to promote connective tissue deposition, angiogenesis, and fibroplasia for repairing a tissue defect. U.S. Pat. No. 6,284,284 to Naughton discloses compositions for the repair of skin defects using natural human extracellular matrix by injection, entire contents of which are incorporated herein by reference. Bandman et al. in U.S. Pat. No. 6,303,765 discloses human extracellular matrix protein and polynucleotides which identify and encode the matrix protein, wherein the human extracellular matrix protein and its polynucleotides may form a shapeable biomaterial of the present invention.

The shapeable polymer as a biomaterial in the present invention may also comprise biodegradable polymer and non-biodegradable polymer, including prepolymer and polymer suspension. In one embodiment, the shapeable polymer in this invention may be selected from a group consisting of silicone, polyurethane, polyamide, polyester, polystyrene, polypropylene, polyacrylate, polyvinyl, polycarbonate, polytetrafluoroethylene, poly (l-lactic acid), poly (d, l-lactide glycolide) copolymer, polyorthoester, polycaprolactone, poly (hydroxybutyrate/hydroxyvaleerate) copolymer, nitrocellulose compound, polyglycolic acid, cellulose, gelatin, dextran, and combination thereof.

Slepian et al. in U.S. Pat. No. 5,947,977 discloses a novel process for paving or sealing the interior surface of a tissue lumen by entering the interior of the tissue lumen and applying a polymer to the interior surface of the tissue lumen. Slepian et al. further discloses that the polymer can be delivered to the lumen as a monomer or prepolymer solution, or as an at least partially preformed layer on an expansible member, the entire contents of which are incorporated herein by reference. The polymer as disclosed may be suitable as a component of the shapeable biomaterial of the present invention.

A method for joining or restructuring tissue consisting of providing a preformed sheet or film which fuses to tissue upon the application of energy is disclosed in U.S. Pat. No. 5,669,934, entire contents of which are incorporated herein by reference. Thus, the protein elements of the tissue and the collagen filler material can be melted or denatured, mixed or combined, fused and then cooled to form a weld joint. However, the heat shapeable biomaterial of the present invention may comprise collagen matrix configured and adapted for in vivo administration by injection via a catheter system at a site of the tissue defect; and applying heat sufficient to shape the biomaterial and immobilize the biomaterial at about the tissue defect, but not to weld the tissue.

An injectable bulking agent composed of microspheres of crosslinked dextran suspended in a carrier gel of stabilized hyaluronic acid is marketed by Q-Med AB (Uppsala, Sweden). In one embodiment of applications, this dextran product may be injected submucosally in the urinary bladder in close proximity to the ureteral orifice. The injection of dextran creates increased tissue bulk, thereby providing coaptation of the distal ureter during filling and contraction of the bladder. The dextran microspheres are gradually surrounded by body's own connective tissue, which provides the final bulking effect. The heat shapeable polymer of the present invention may comprise dextran configured and adapted for in vivo administration by injection via a catheter system at a site of the tissue defect; and applying heat sufficient to shape the biomaterial and immobilize the biomaterial at about the tissue defect.

Sinofsky et al. in U.S. Pat. No. 5,100,429 discloses an uncured or partially cured, collagen-based material delivered to a selected site in a blood vessel and is crosslinked to form an endoluminal stent, entire contents of which are incorporated herein by reference. The collagen-based material as disclosed may form a component of the shapeable biomaterial of the present invention.

Edwards in PCT WO 01/52930 discloses a method and system for shrinking dilatations of a body, removing excess, weak or diseased tissues and strengthening remaining tissue of the lumen walls, the entire contents of which are incorporated herein by reference. However, Edwards does not disclose a method for repairing a tissue defect comprising: injecting a heat shapeable biomaterial formulated for in vivo administration by injection via a percutaneous delivery system at a site of the tissue defect; and applying heat to the biomaterial and a portion of the tissue defect adapted for shaping the biomaterial, the heat being below a temperature sufficient for effecting crosslinking of the biomaterial and the portion of the tissue defect.

Therefore, it is a further embodiment to provide a method for repairing a tissue defect comprising: injecting a heat shapeable biomaterial formulated for in vivo administration by injection via a percutaneous delivery system at a site of the tissue defect; and applying heat to the biomaterial and a portion of the tissue defect adapted for shaping the biomaterial, the heat being below a temperature sufficient for effecting crosslinking of the biomaterial and the portion of the tissue defect, the tissue defect may comprise vulnerable plaque, calcified tissue, or other lesions of atherosclerosis.

From the foregoing, it should now be appreciated that an improved delivery system and methods having needle electrode means for injecting shapeable biomaterial and high frequency current energy for penetrating the tissue of a valvular annulus in order to tighten and stabilize the shapeable biomaterial inside an annular organ structure has been disclosed for repairing an annular organ structure of a heart valve, an annular organ structure of a venous valve, a valve leaflet, chordae tendinae, papillary muscles, and the like. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

What is claimed is:

1. A method for repairing a valvular annulus defect comprising: injecting a heat shapeable biomaterial formulated for in vivo administration by injection via a delivery system at a site of the valvular annulus defect; and applying heat sufficient to shape said biomaterial and immobilize said biomaterial at about said annulus defect.

2. The method of claim 1, wherein said biomaterial is a matrix of collagen.

3. The method of claim 2, wherein said heat is provided as a temperature below a temperature for effecting crosslinking of said biomaterial.

4. The method of claim 1, wherein said biomaterial further comprises a pharmaceutically acceptable carrier for treating the annulus defect and a drug is loaded with said pharmaceutically acceptable carrier.

5. The method of claim 4, wherein the drug is selected from a group consisting of an anti-clotting agent, an anti-inflammatory agent, an anti-virus agent, an antibiotics, a tissue growth factor, an anesthetic agent, a regulator of angiogenesis, a steroid, and combination thereof.

6. The method of claim 1, wherein said biomaterial is a connective tissue protein comprising naturally secreted extracellular matrix.

7. The method of claim 1, wherein said biomaterial is a heat shapeable polymer.

8. The method of claim 7, wherein said heat shapeable polymer is selected from a group consisting of polyamide, polyester, polystyrene, polypropylene, polyacrylate, polyvinyl, polycarbonate, polytetrafluoroethylene, poly (l-lactic acid), poly (d, l-lactide glycolide) copolymer, polyorthoester, polycaprolactone, poly (hydroxybutyrate/hydroxyvaleerate) copolymer, nitrocellulose compound, polyglycolic acid, cellulose, gelatin, dextran, and combination thereof.

9. The method of claim 1, wherein the valvular annulus is selected from a group consisting of a mitral valve, a tricuspid valve, a pulmonary valve, an aortic valve, and a venous valve.

10. The method of claim 1, wherein said delivery system comprises:
a flexible catheter shaft having a distal tip section, a distal end, a proximal end, and at least one lumen extending between the distal end and the proximal end;
a flexible tissue-contactor ring located at the distal tip section and inside the at least one lumen of said catheter shaft for contacting an inner wall of the valvular annulus defect, wherein said tissue-contactor ring is deployable out of the at least one lumen by a tissue-contactor deployment mechanism and is preformed to have an appropriate shape compatible with said inner wall of the valvular annulus defect, wherein said appropriate shape is a circular shape, a D-shape, a kidney shape, or an oval shape;
a needle electrode element located at or within the flexible tissue-contactor ring for penetrating into a tissue, wherein the needle electrode element is deployable out of the tissue-contactor ring in a manner essentially perpendicular to a longitudinal axis of the catheter shaft;
a handle attached to the proximal end of the catheter shaft, wherein the handle comprises the tissue-contactor deployment mechanism and an electrode deployment means for advancing the needle electrode out of said tissue-contactor ring; and
a high frequency current generator, wherein an electrical conductor means for transmitting high frequency current to said needle electrode element is provided.

11. The method of claim 10, wherein the tissue-contactor ring is made of a biocompatible material selected from a group consisting of silicone, latex, polyurethane, fabric, and a combination thereof.

12. The method of claim 10, wherein the tissue-contactor ring comprises a plurality of open channels for a fluid to pass from a proximal end of said tissue-contactor ring to a distal end of said tissue-contactor ring.

13. The method of claim 10 further comprising:
(a) percutaneously introducing the delivery system through a blood vessel to a site of the valvular annulus or introducing the delivery system through a thoroscopy port into a heart or injecting said heat shapeable biomaterial during an open heart surgery;
(b) positioning the tissue-contactor ring of the catheter shaft on the inner wall of the valvular annulus;
(c) advancing the needle electrode element for penetrating the needle electrode element into a tissue of the valvular annulus;
(d) injecting heat shapeable biomaterial at the site of the valvular annulus defect; and
(e) applying high frequency current through the electrical conductor means to the needle electrode element for repairing the valvular annulus defect.

14. The method of claim 13, wherein the needle electrode element comprises a plurality of needle electrodes that are preshaped to be essentially perpendicular to a longitudinal axis of the catheter shaft when deployed and wherein the high frequency current is delivered to each of said plurality of needle electrodes in a mode selected from a group consisting of individual mode, pulsed mode, sequential mode, and simultaneous mode.

15. The method of claim 10, wherein the high frequency current is selected from a group consisting of radiofrequency current, microwave current and ultrasound current.

16. A method for repairing a valvular annulus defect comprising: injecting a heat shapeable biomaterial formulated for in vivo administration by injection via a delivery system at a site of the valvular annulus defect; and applying heat sufficient to shape tissue surrounding said annulus defect and said biomaterial and immobilize said biomaterial at about said annulus defect.

17. A method for repairing a tissue defect comprising: injecting a heat shapeable biomaterial formulated for in vivo administration by injection via a percutaneous delivery system at a site of the tissue defect; and applying heat to said biomaterial and a portion of the tissue defect adapted for shaping said biomaterial, said heat being below a temperature sufficient for effecting crosslinking of said biomaterial and the portion of the tissue defect.

18. The method of claim 17, wherein heat is provided by a high frequency current source selected from a group consisting of radiofrequency current, microwave current, and ultrasound current.

19. The method of claim 17, wherein the tissue defect comprises vulnerable plaque, calcified tissue, or other lesions of atherosclerosis.

20. The method of claim 17, wherein the biomaterial comprises a matrix of collagen.
